(19) United States
(12) Patent Application Publication
(10) Pub. No.: US 2024/0358266 A1
(43) Pub. Date: Oct. 31, 2024

(54) SYSTEMS AND METHOD FOR MONITORING PENILE BLOOD FLOW DURING SURGERY

(71) Applicant: Neuro-Vascular Research and Design Corporation, Tucson, AZ (US)

(72) Inventors: Jeffrey Owen, Tucson, AZ (US); Nicholas Chung, New York, NY (US); H. Shields Day, Cohasset, MA (US); Obed Espina, Brooklyn, NY (US)

(21) Appl. No.: 18/643,493

(22) Filed: Apr. 23, 2024

Related U.S. Application Data
(60) Provisional application No. 63/461,557, filed on Apr. 24, 2023.

(51) Int. Cl. A61B 5/026; A61B 5/00; A61B 5/145; A61B 5/1455; A61B 5/391; A61B 5/395; A61B 5/397

(52) U.S. Cl. CPC A61B 5/0261; A61B 5/14546; A61B 5/1455; A61B 5/391; A61B 5/395; A61B 5/397; A61B 5/6813; A61B 5/6832; A61B 5/6838; A61B 5/7267; A61B 5/7405; A61B 5/742; A61B 5/746; A61B 2560/0204; A61B 2560/045; A61B 2560/0462; A61B 2562/0233; A61B 2562/227

(57) ABSTRACT

Exemplary embodiments provide an optical penile blood flow measuring technology for the monitoring of neurological and vascular function of structures at surgical risk. The measuring principle is to rely on optical absorption properties of blood components for penile blood flow monitoring. Certain embodiments use different wavelengths to measure changes in hemoglobin concentrations and are particularly advantageous in continuous, autonomous monitoring with real-time feedback to surgeons for potential intervention and decision-making in a timely manner. The measurements results can be used to provide real-time feedback to surgeons operating on the patient, or for off-line analysis and evaluation. Certain embodiments can operate autonomously in the continuous measurement mode, a synchronized mode, or a triggered mode, reducing the need of highly-skilled apparatus operators, allowing automatic and real-time warning to surgeons when operating in areas while preserving the functions of critical nerves, resulting in improved clinical outcomes and reducing operation costs.

### CROSS-REFERENCE TO RELATED APPLICATION(S)

This patent application claims the benefit of U.S. Provisional Patent Application No. 63/461,557 entitled OPTICAL PENILE BLOOD FLOW MEASURING AND RECORDING SYSTEM CONFIGURED TO MONITOR NEUROLOGICAL AND VASCULAR FUNCTION OF STRUCTURES AT SURGICAL RISK filed Apr. 24, 2023, which is hereby incorporated herein by reference in its entirety.

### FIELD OF THE INVENTION

The invention generally relates to continuous recording of penile blood flow during surgery.

### BACKGROUND OF THE INVENTION

A radical prostatectomy is surgery to remove the prostate gland and seminal vesicles (and sometimes nearby lymph nodes) after a prostate cancer diagnosis. According to cancer.org, in the more traditional approach to prostatectomy, called an open prostatectomy, the surgeon operates through a single long skin incision (cut) to remove the prostate and nearby tissues. This type of surgery is done less often than in the past. In a laparoscopic prostatectomy, the surgeon makes several smaller incisions and uses special long surgical tools to remove the prostate. The surgeon either holds the tools directly, or uses a control panel to precisely move robotic arms that hold the tools. This approach to prostatectomy has become more common in recent years. If done by experienced surgeons, the laparoscopic radical prostatectomy can give results similar to the open approach.

The major possible side effects of radical prostatectomy are urinary incontinence (being unable to control urine) and erectile dysfunction (impotence; problems getting or keeping erections). These side effects can also occur with other forms of prostate cancer treatment. Urinary incontinence: You may not be able to control your urine or you may have leakage or dribbling. Being incontinent can affect you not only physically but emotionally and socially as well. These are the major types of incontinence:
(a) Men with stress incontinence might leak urine when they cough, laugh, sneeze, or exercise. Stress incontinence is the most common type after prostate surgery. It's usually caused by problems with the valve that keeps urine in the bladder (the bladder sphincter). Prostate cancer treatments can damage this valve or the nerves that keep the valve working.
(b) Men with overflow incontinence have trouble emptying their bladder. They take a long time to urinate and have a dribbling stream with little force. Overflow incontinence is usually caused by blockage or narrowing of the bladder outlet by scar tissue.
(c) Men with urge incontinence have a sudden need to urinate. This happens when the bladder becomes too sensitive to stretching as it fills with urine.
(d) Rarely after surgery, men lose all ability to control their urine. This is called continuous incontinence.

After surgery for prostate cancer, normal bladder control usually returns within several weeks or months. This recovery usually occurs slowly over time. Doctors can't predict for sure how any man will be affected after surgery. In general, older men tend to have more incontinence problems than younger men. Large cancer centers, where prostate surgery is done often and surgeons have a lot of experience, generally report fewer problems with incontinence. Incontinence can be treated. Even if incontinence can't be corrected completely, it can still be helped.

Erectile dysfunction (impotence): This means the patient can't get an erection sufficient for sexual penetration. Erections are controlled by two tiny bundles of nerves that run on either side of the prostate. If a patient can have erections before surgery, the surgeon will try not to injure these nerves during the prostatectomy. This is known as a nerve-sparing approach. But if the cancer is growing into or very close to the nerves, the surgeon will need to remove them. If both nerves are removed, the patient won't be able to have spontaneous erections but still might be able to have erections using some of the aids described below. If the nerves on only one side are removed, the patient still might have erections, but the chance is lower than if neither were removed. If neither nerve bundle is removed, the patient might have normal erections at some point after surgery. The ability to have an erection after surgery depends on factors including age, ability to get an erection before the operation, and whether the nerves were cut. All men can expect some decrease in the ability to have an erection, but the younger the patient, the more likely it is that the patient will keep this ability. Surgeons who do many radical prostatectomies tend to report lower impotence rates than doctors who do the surgery less often. A wide range of impotency rates have been reported in the medical literature, but each man's situation is different, so the best way to get an idea of your chances for recovering erections is to ask about your doctor's success rates and what the outcome is likely to be in your case. If your ability to have erections does return after surgery, it often returns slowly. In fact, it can take from a few months up to 2 years. During the first few months, you will probably not be able to have a spontaneous erection, so you may need to use medicines or other treatments. Most doctors feel that regaining potency is helped along by trying to get an erection as soon as possible once the body has had a chance to heal (usually several weeks after the operation). Some doctors call this penile rehabilitation. Medicines (see below) may be helpful at this time. Be sure to talk to your doctor about your situation.

There are several options for treating erectile dysfunction:
(a) Phosphodiesterase-5 (PDE5) inhibitors such as sildenafil (Viagra), vardenafil (Levitra), tadalafil (Cialis), and avanafil (Stendra) are pills that can help with erections. These drugs won't work if both nerves that control erections have been damaged or removed. Common side effects of these drugs are headache, flushing (skin becomes red and feels warm), upset stomach, light sensitivity, and runny or stuffy nose. Rarely, these drugs can cause vision problems, possibly even blindness. Some other drugs such as nitrates, which are drugs used to treat heart disease, can cause problems if you are taking a PDE5 inhibitor, so be sure your doctor knows what medicines you take.
(b) Alprostadil is a man-made version of prostaglandin El, a substance naturally made in the body that can produce erections. It can be injected almost painlessly into the base of the penis 5 to 10 minutes before intercourse or placed into the tip of the penis as a suppository. You can even increase the dosage to prolong the erection. You might have side effects, such as pain, dizziness, and prolonged erection, but they are not usually serious.
(c) Vacuum devices are another option to create an erection. These mechanical pumps are placed over the penis. The air is sucked out of the pump, which draws blood into the penis to produce an erection. The erection is maintained after the pump is removed by a strong rubber band placed at the base of the penis. The band is removed after sex.
(d) Penile implants might restore your ability to have erections if other methods don't help. An operation is needed to put them inside the penis. There are several types of penile implants, including those using silicone rods or inflatable devices.

Changes in orgasm: After surgery, the sensation of orgasm should still be pleasurable, but there is no ejaculation of semen—the orgasm is “dry.” This is because the glands that made most of the fluid for semen (the seminal vesicles and prostate) were removed during the prostatectomy, and the pathways used by sperm (the vas deferens) were cut. In some men, orgasm becomes less intense or goes away completely. Less often, men report pain with orgasm.

Loss of fertility: Radical prostatectomy cuts the vas deferens, which are the pathways between the testicles (where sperm are made) and the urethra (through which sperm leave the body). Your testicles will still make sperm, but they can't leave the body as a part of the ejaculate. This means that a man can no longer father a child the natural way. Often, this is not an issue, as men with prostate cancer tend to be older. But if it is a concern for you, you might want to ask your doctor about “banking” your sperm before the operation. To learn more, see Fertility and Men With Cancer.

Lymphedema: This is a rare but possible complication of removing many of the lymph nodes around the prostate. Lymph nodes normally provide a way for fluid to return to the heart from all areas of the body. When nodes are removed, fluid can collect in the legs or genital region over time, causing swelling and pain. Lymphedema can usually be treated with physical therapy, although it may not go away completely. You can learn more on our lymphedema page.

Change in penis length: A possible effect of surgery is a small decrease in penis length. This is probably due to a shortening of the urethra when a portion of it is removed along with the prostate.

Inguinal hernia: A prostatectomy increases a man's chances of developing an inguinal (groin) hernia in the future.

### SUMMARY OF VARIOUS EMBODIMENTS

In accordance with one embodiment of the invention, an optical penile structure function monitoring system comprises a measuring probe configured to be secured via an attachment mechanism to a penis of a patient during and over tumescence and flaccid penile events and further comprises a control system having a first detector that detects penile blood flow based on signals received from the measuring probe and a second detector that detects tumescence and flaccid penile events based on signals received from the first detector.

In accordance with various alternative embodiments, the measuring probe May include at least one optode assembly of optical components to measure penile blood flow hemodynamics. The measuring probe may include a compressible optically non-reflective mask to separate at least two optical components. The measuring probe may be configured to convert optical signals to electrical signals, and the control system may be configured to process the electrical signals from the measuring probe and to record continuous raw data and penile function data. The measuring probe may include a synchronization mechanism to allow the sharing of optical components. The attachment mechanism may be configured to adapt to different penile sizes using the same hardware. The attachment mechanism may be secured, for example, using a penile clip and/or double-sided tape.

In additional alternative embodiments, the measuring probe and control system may be configured to measure optical absorbance signals with at least one wavelength, e.g., two wavelengths such as red and near infrared wavelengths that can be used as the probing light for oxyhemoglobins and deoxyhemoglobins that have opposite relative optical absorbance between the two wavelengths. The measurements may measurements alternate between the red and near infrared wavelengths, e.g., at a fixed rate. The control system may be configured to provide in-time warnings to surgeons operating on a patient who might have their penile function at risk based on the signals received from the measuring probe. The control system may include a control unit separate from the measuring probe, where, for example, communication between the control unit and the measuring probe may comprise flexible shielded cables or a wireless connection.

In additional alternative embodiments, normal ranges of variability, thresholds, and types of significant data changes as a function of surgical maneuver or other physiological parameters are established and stored. At least one of (a) the normal ranges, thresholds, and types of significant data changes are established using machine learning, or (b) the in-time warnings may be generated using machine learning. The in-time warnings may include at least one of a visually warning or an audible warning. The control system may include a memory to record any warnings and their associated measurements. At least one of the measuring probe or the control system may be configured to provide visual guidance to configure the system according to the surgery type.

In additional alternative embodiments, the system also may include an EMG monitor that measures muscle response or electrical activity in response to stimulation of a nerve of the patient, wherein the control system is configured to monitor the intensity and speed of the change in penile blood flow to develop pathological patterns and identify nerve irritation and vascular irritation in real-time when combined with signals from the EMG monitor. The control system may be further configured to develop pathological patterns and identify nerve irritation and vascular irritation in real-time using machine learning.

In additional alternative embodiments, the measuring probe may include at least one LED capable of producing red wavelength light and near infrared wavelength light and further comprises at least one photodiode capable of measuring red wavelength light and near infrared wavelength light. The at least one LED may include at least one red wavelength LED and at least one near infrared wavelength LED. The at least one photodiode may include at least one red wavelength photodiode and at least one near infrared photodiode or may include a single photodiode used to detect both the red wavelength light and the near infrared wavelength light. The at least one photodiode may include a first photodiode placed closer to the red and near infrared LEDs for detecting light that probes a shallower region and a second photodiode placed further from the red and near infrared LEDs for detecting light that probes a deeper region. The measuring probe may include at least one optically non-reflecting mask configured to prevent undesired optical cross-interference between the at least one LED and the at least one photodiode and further configured to ensure that probing light from the at least one LED does not have a path for reaching the at least one photodiode without going through the probed tissue first. The measuring probe may be configured or controllable to alternate between producing red wavelength light and producing near infrared wavelength light.

In accordance with another embodiment of the invention, a measuring probe may include at least one LED capable of producing red wavelength light and near infrared wavelength light and also may include at least one photodiode capable of measuring red wavelength light and near infrared wavelength light.

In various alternative embodiment, the at least one LED may include at least one red wavelength LED and at least one near infrared wavelength LED. The at least one photodiode may include at least one red wavelength photodiode and at least one near infrared photodiode or may include a single photodiode used to detect both the red wavelength light and the near infrared wavelength light. The at least one photodiode may include at least a first photodiode placed closer to the red and near infrared LEDs for detecting light that probes a shallower region and a second photodiode placed further from the red and near infrared LEDs for detecting light that probes a deeper region. The measuring probe may include at least one optically non-reflecting mask configured to prevent undesired optical cross-interference between the at least one LED and the at least one photodiode and further configured to ensure that probing light from the at least one LED does not have a path for reaching the at least one photodiode without going through the probed tissue first. The measuring probe may be configured or controllable to alternate between producing red wavelength light and producing near infrared wavelength light. The measuring probe may include an attachment mechanism configured to secure the measuring probe to a penis of a patient during and over tumescence and flaccid penile events and may be configured to adapt to different penile sizes using the same hardware. The attachment mechanism may include a penile clip and/or double-sided tape. The measuring probe also may include a communication interface for wired communication with a control system and/or a communication interface for wireless communication with a control system. The measuring probe may include a synchronization mechanism to allow the sharing of optical components.

In accordance with another embodiment of the invention, a method for monitoring a patient comprises monitoring penile blood flow of the patient; determining a normal range of penile blood flow for the patient; initiating nerve stimulation to elicit tumescence; applying a machine learning-trained algorithm to identify and classify penile blood flow in response to the nerve stimulation; and initiating intervention when the penile blood flow in response to the nerve stimulation is outside of the normal range of penile blood flow for the patient.

In various alternative embodiments, monitoring penile blood flow of the patient may include monitoring blood oxygenation data, and initiating nerve stimulation may use a stimulating electrode.

Additional embodiments may be disclosed and claimed.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 shows a preferred embodiment of the electronic portion of the optode

FIG. 2 shows a side view of the preferred embodiment of the electronic portion of the optode as mentioned previously in FIG. 1.

FIG. 3 shows one embodiment of the optode holder.

FIG. 4 shows a preferred embodiment of the optode assembly in the form of a clip.

FIG. 5 shows a block diagram representation of a control unit.

FIG. 6 shows the absorption data of changes in deoxyhemoglobin relative concentration in penile blood.

FIG. 7 shows the absorption data of changes in oxyhemoglobin relative concentration in penile blood.

FIG. 8 shows the overlay of three normalized test runs.

FIG. 9 shows a data flow diagram representation of the Sentinel system in accordance with certain embodiments.

FIG. 10 shows a data flow diagram of the Sentinel evaluation methodology decision tree in accordance with certain embodiments.

It should be noted that the foregoing figures and the elements depicted therein are not necessarily drawn to consistent scale or to any scale. Unless the context otherwise suggests, like elements are indicated by like numerals. The drawings are primarily for illustrative purposes and are not intended to limit the scope of the inventive subject matter described herein.

### DESCRIPTION OF ILLUSTRATIVE EMBODIMENTS

### Introduction

Certain embodiments provide continuous recording of penile blood flow such as during surgery. For convenience, the system is referred to herein as the Sentinel™ system although it should be noted that embodiments are not limited to implementation in any particular commercial or non-commercial product.

Current devices are very large and must be held in place by a technologist, which makes them impractical for use during surgery. The reliability of our data is expected to be superior to that of a traditional handheld device. By using miniaturization and automation, we can monitor blood flow continuously for the duration of surgery. Because the device is fixed in place, skin contact will remain stable, and data will be more reliable.

Exemplary embodiments use miniaturized sensors and optodes and appropriate software to automatically recording blood flow continuously during surgery without the need for testing personnel. The claimed invention differs from what currently exists. Our monitoring optodes and sensors are miniaturized and don't require personnel to hold them in place. The device can be held in place on the penis without interfering with the sterile field. From a practical perspective, it is impossible for a technologist to hold a testing probe in place continuously during a surgery, especially in a sterile location. Thus, data from a hand-held device is likely to vary due to changes in skin contact, which will reduce data reliability. By using miniaturization and automation we can monitor blood flow continuously for the duration of surgery. Because the device is fixed in place, skin contact will remain stable and data will be more reliable.

Exemplary embodiments provide an optical penile blood flow measuring technology for the monitoring of neurological and vascular function of structures at surgical risk. The measuring principle is to rely on optical absorption properties of blood components for penile blood flow monitoring. The preferred embodiment uses different wavelengths to measure changes in hemoglobin concentrations. Blood flow is expected to increase during the transition from flaccid state to tumescence state resulting in an increase in oxyhemoglobin concentrations. Similarly, during the same time, deoxyhemoglobin concentration is expected to decrease. On the other hand, when the penis returns to flaccid state from tumescence state, the opposite trends are expected. In other embodiment, other blood related parameters can also be used for the purpose of monitoring penile blood flow. This disclosure is particularly advantageous in continuous, autonomous monitoring with real-time feedback to surgeons for potential intervention and decision-making in a timely manner. The measurements results can be used to provide real-time feedback to surgeons operating on the patient, or for off-line analysis and evaluation. The preferred embodiment can operate autonomously in the continuous measurement mode, a synchronized mode, or a triggered mode, reducing the need of highly-skilled apparatus operators, allowing automatic and real-time warning to surgeons when operating in areas while preserving the functions of critical nerves, resulting in improved clinical outcomes and reducing operation costs.

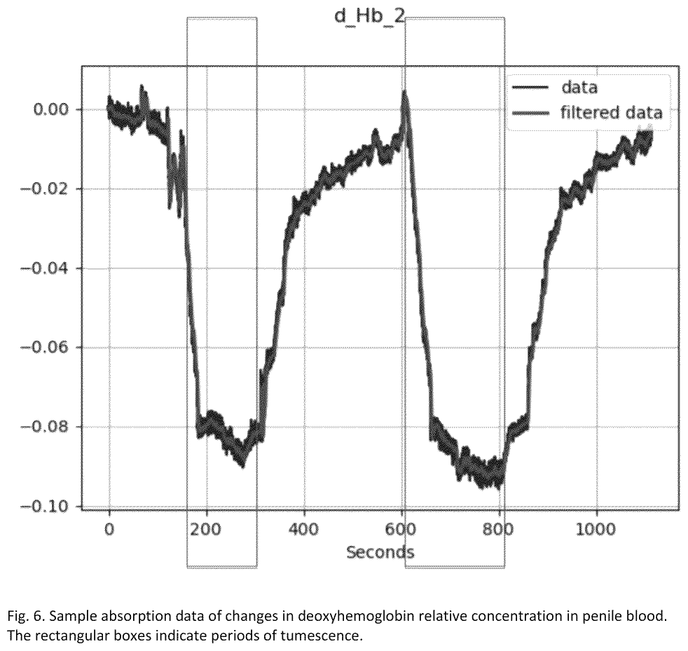

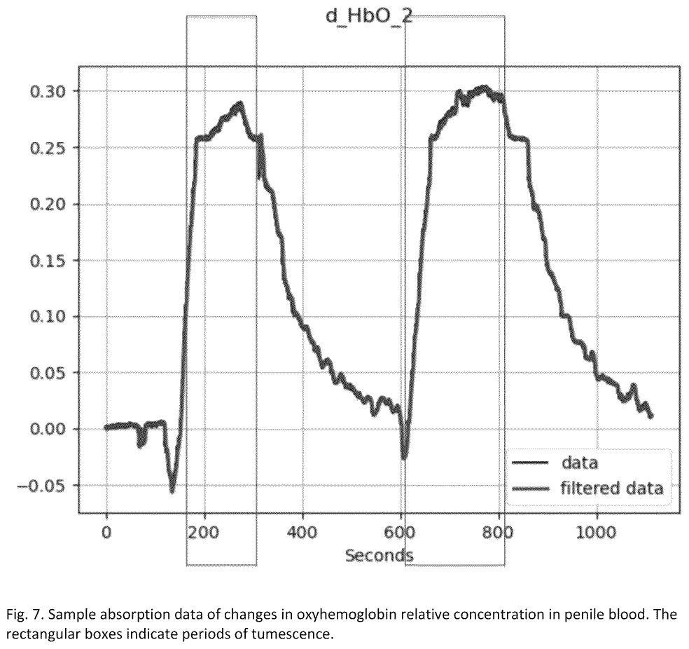

Data collected using the disclosed invention demonstrates its feasibility and reproducibility. FIG. 6 shows the absorption data of changes in deoxyhemoglobin relative concentration in penile blood. The rectangular boxes indicate periods of tumescence. FIG. 7 shows the absorption data of changes in oxyhemoglobin relative concentration in penile blood. The rectangular boxes indicate periods of tumescence.

The device preferably will be placed on the dorsal side of the penis, near its base. The device will be held in place with a medical grade double-sided sticky tape. Once in place elastic non-sticky tape will be wrapped around the penis, overlapping the device. This tape will further hold the device in place and prevent extraneous light from entering the sensors. By using a miniaturized sensor, double sided sticky tape, and elastic non-sticky tape the device is held in place without the need for human intervention.

### Hardware

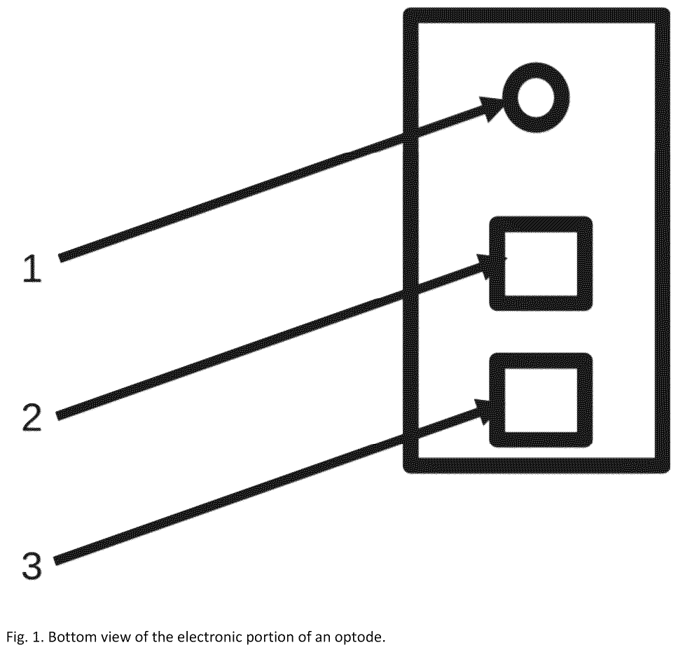

Hardware components for certain embodiments are described with reference to FIGS. 1-5 in which the following reference numerals may be used:
1. Optical sources (top view of the component)
2. Optical detector A
3. Optical detector B
4. Optical sources (side view of the component)
5. Optical detector A
6. Optical detector B
7. Communication unit
8. Signal conditioning unit A
9. Signal conditioning unit B
10. Board or substrate (e.g., printed circuit board (PCB))
11. Optical detector A (e.g., Photodiode A)
12. Optical detector B (e.g., Photodiode B)
13. Signal conditioning unit A (e.g., Amplifiers)
14. Optical sources (e.g., LED)
15. Communication unit (e.g., a wireless module)
16. Mask openings
17. Optode holder
18. Spring slot A
19. Spring slot B
20. Optically non-reflecting mask
21. Optode circuit
22. Attachment mechanism (e.g., Chain)
23. Control unit
24. Control, data processing, and communication electronics
25. Power unit (e.g., Battery pack)
26. Cable
27. Clip left leg
28. Embedded torsion springs
29. Clip right leg
30. Bridge
31. LED driver
32. Optical detector
33. Power management
34. Wireless module
35. Timing circuit
36. Communication protocol
37. Control unit

The following are additional details of the reference numerals in accordance with certain embodiments:
1. Optical source can emit two wavelengths (e.g., red and near-infrared) and is near the optical detector
2. Optical detector is in closer proximity to the optical source; with the other detector(s), comprises the detector array
3. Optical detector that is further away from the optical source, relative to the other(s)
4. Optical sources, or an array comprised of multiple optical sources
5. Optical detector in alternate embodiment
6. Optical detector in alternate embodiment
7. Communication unit enables optode to communicate with the control unit or other sensing modules; is wired or wireless
8. Signal conditioning unit transimpedance amplifiers to convert the currents from the photodiodes to voltages, amplify, and condition the output signal
9. Signal conditioning unit can be combined with another unit to comprise a single component
10. Board or substrate, in this embodiment a printed circuit board, to which all components are affixed
11. Photodiode measures the probing light that has gone through the probed tissue
12. Photodiode, along with other photodiodes, each represents a component of the optical detectors
13. Amplifier converts and amplifies the signal from the photodiode and can also optionally serve the function of signal transduction
14. One or more LEDs produce probing light of single or multiple wavelengths that will go through the tissue being measured
15. Communication unit transmits the data signal from the amplifier
16. Openings in the optically non-reflecting mask enable probing light to be directed at the tissue being measured while blocking internal, external, or intrinsic interfering light
17. Holder is attached to the rest of the optode assembly and can be enabled to firmly and gently adapt to the varying dimension of a penis via a spring mechanism
18. Spring fits into slot designed to hold spring in place and secure optode assembly to penis
19. Spring fits into slot, as with previous spring
20. Optically non-reflecting and opaque or absorbing mask ensure there is no undesired optical cross-interference among the photodiodes and no external or intrinsic interference
21. Optode circuit contains the LEDs, amplifiers, the communication unit, and related components
22. Chain or similar looping mechanism attached to the side of the control unit to allow the unit to be hung
23. Control unit, or alternatively, the control box, connects to the optode assembly by cable, and houses the control, battery, data processing, and communication electronics
24. Control, data processing, communication electronics, and battery that comprise and enable the main functioning of the control unit
25. Power unit, such as a battery pack, enables battery operation of the control unit and optode assembly
26. Cable transmits analog or digital signals between the control unit and optode assembly
27. Clip, the left of two that comprise the optode assembly
28. Embedded torsion springs in the bridge connect the two clip legs
29. Clip, the right of two that comprise the optode assembly
30. Bridge connects the two clip legs with embedded torsion springs
31. LED driver controls the timing of the red and near infrared LEDs
32. Optical detector converts the analog optical signal coming from the optode assemblies into digital signal
33. Power management sources from the available power source, e.g., a rechargeable battery, and regulates and conditions the power before supplying it
34. Wireless module provides a means to wirelessly communicate with external devices
35. Timing circuit allows synchronization of the various modules and proper allocation of resources to the various clients served
36. Communication protocol ensures proper communication to take place, establishes secure connections when necessary, and allows proper authorizations
37. Control unit embodiment that houses the LED driver, optical detector, power management, wireless module, timing circuit, and communication protocol.

In certain embodiments of the apparatus, the measuring principle is to rely on optical properties of blood components for penile blood flow monitoring. The apparatus, when turned on, can be connected to a measurement station to record the data it produces. To begin measurement, the apparatus is affixed to the subject's penis, preferably the proximal portion, with the optical arrays aligned over the side of the penis (targeting the Corpora Cavernosa regions), and is powered on. When the subject experiences stimulation, including, but not limited to visual sexual stimulation (VSS), sacral nerve stimulation, or another form of manual stimulation, arteries are expected to be dilated, resulting in the filling of the Corpora Cavernosa with blood while veins are compressed resulting in the restriction of outward blood flow. The above-mentioned mechanism is expected to 1) increase the total hemoglobin, 2) increase blood flow and oxyhemoglobin concentrations during the transition from flaccid state to tumescence state. Similarly, during the same time, deoxyhemoglobin concentration is expected to decrease. It is noted that depending on the exact locations of the probing and the timing of the measurement, the measured hemodynamics can appear to vary. In our implementation, all hemodynamic parameters both explicitly mentioned and implicitly referenced are used to ensure quality and reliability of our measurement. The apparatus produces a filtered and amplified signal that is transmitted to the processing station. When the penis returns to flaccid state from tumescence state, the opposite trends are expected. The measurement results can then be used to provide real-time feedback (i.e., to surgeons operating on the subject), or for off-line analysis and evaluation.

The following disclosure is for an optical penile blood flow measuring technology for the monitoring of neurological and vascular function of structures at surgical risk. The measuring principle is to rely on optical properties of blood components for penile blood flow monitoring. The preferred embodiment uses different wavelength(s) to measure changes in hemoglobin concentrations. When the subject experiences stimulation, including, but not limited to visual sexual stimulation (VSS), sacral nerve stimulation, or another form of manual stimulation, arteries are expected to be dilated, resulting in the filling of the Corpora Cavernosa with blood while veins are compressed resulting in the restriction of outward blood flow. The above-mentioned mechanism is expected to 1) increase the total hemoglobin, 2) increase blood flow and oxyhemoglobin concentrations during the transition from flaccid state to tumescence state. Similarly, during the same time, deoxyhemoglobin concentration is expected to decrease. It is noted that depending on the exact locations of the probing and the timing of the measurement, the measured hemodynamics can appear to vary. In our implementation, all hemodynamic parameters both explicitly mentioned and implicitly referenced are used to ensure quality and reliability of our measurement. In other embodiments, other blood related parameters can also be used for the purpose of monitoring penile blood flow. This disclosure is particularly advantageous in continuous, autonomous monitoring with real-time feedback to surgeons for potential intervention and decision-making in a timely manner. The measurements results can be used to provide real-time feedback to surgeons operating on the patient, or for off-line analysis and evaluation. The preferred embodiment can operate autonomously in the continuous measurement mode, a synchronized mode, or a triggered mode, reducing the need of highly-skilled apparatus operators, allowing automatic and real-time warning to surgeons when operating in areas while preserving the functions of critical nerves, resulting in improved clinical outcomes and reducing operation costs.

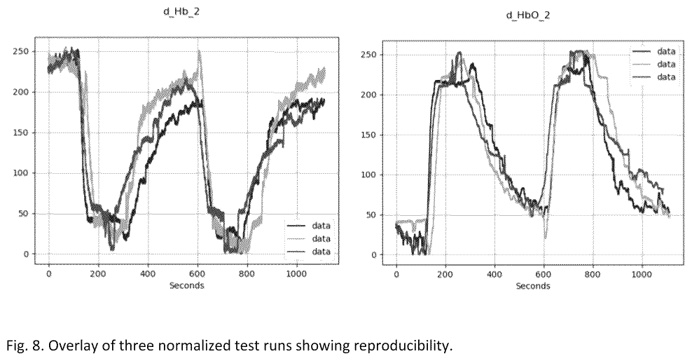

Data collected using the disclosed invention demonstrates its feasibility and reproducibility. FIG. 6 shows the absorption data of changes in deoxyhemoglobin relative concentration in penile blood. The rectangular boxes indicate periods of tumescence. FIG. 7 shows the absorption data of changes in oxyhemoglobin relative concentration in penile blood. The rectangular boxes indicate periods of tumescence. The changes shown in both figures agree with the expectation mentioned previously. FIG. 8 shows the overlay of three normalized test runs. It can be seen in the figure that the hemoglobin concentration changes are reproducible and are advantageous to be used as indicators of penile functions.

In one embodiment of the invention, the apparatus consists of a control unit, an array of optical sources, and an array of optical detectors. In one embodiment, the optical sources can consist of one or more units. In the same or another embodiment, the optical detector can consist of one or more units. In a preferred embodiment, the optical sources and detectors can reside in the same module hereby referred to as an optode.

FIG. 1 shows a preferred embodiment of the electronic portion of the optode. In this embodiment, both the optical source 1 and the detector array consist of first an optical detector 2 that is in closer proximity to the optical source 1 and a second optical detector 3 that is further away from optical source 1. Having the optical source and detector in the same module allows a convenient and inexpensive way to control the relative positions between the optical source(s) and detector(s). The wavelengths of the emitted light by the optical source 1 can be optimized based on the targeted application. In one embodiment, the optical source 1 can emit two wavelengths, for instance, one in the red and the other in the near-infrared range. The choice of the wavelengths can allow different probing depths and leveraging different optical absorbance that allow certain biological tissues to be identified, reducing interference and noise, etc. In the preferred embodiment, two different wavelengths in the red and near-infrared are selected to allow better monitoring of blood flow due to oxy-hemoglobin and deoxy-hemoglobin having different optical absorbance for the two wavelengths. The number of optical detectors were limited to two to optimize the size and cost of the optode. In another embodiment, four optical detectors with varying distances to the light source are used to control the probing distances into the tissue and the availability of potentially noise canceling channels.

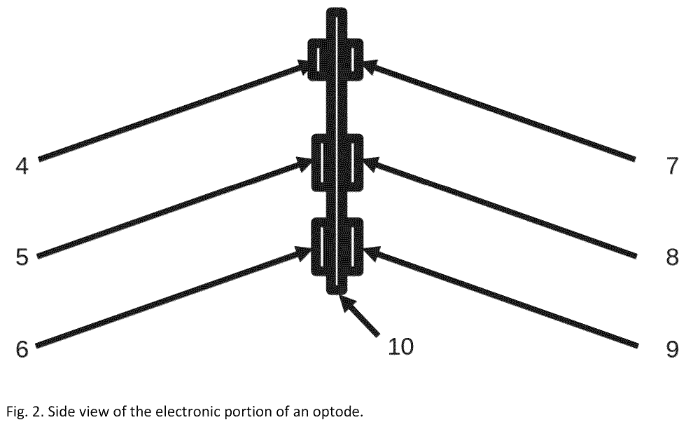

FIG. 2 shows a side view of the preferred embodiment of the electronic portion of the optode as mentioned previously in FIG. 1. Referring to the figure, there are two sides to the optode. The board or substrate 10 is a regular rigid printed circuit board (PCB). In other embodiments, the substrate can be a flexible substrate. On the one side, there are the optical sources 4 and the optical detectors 5, 6. On the other side, there is a communication unit 7 and two signal conditioning units 8, 9. In one embodiment, these can be transimpedance amplifiers to convert the currents from the photodiodes to voltages, amplify and condition the output signal. In another embodiment, the amplifiers can be combined into a single component. In other embodiments, the amplifiers can be a representation of a system of amplifiers serving purposes of cleaning, filtering, amplifying, or any arrangement of the above functions performed on the signals. The signal conditioning units 8, 9 can further include separate filters, filter banks, or other signal conditioning technologies known to persons skilled in the art.

Referring to FIG. 2, the communication unit 7 is for the optode to communicate with the control unit or other sensing modules including other optodes in the system. Other sensing modules can include but are not limited to an electromyography (EMG) or other physiological parameter detecting modules. In a preferred embodiment, an EMG module is connected to the system to augment and enhance the penile function monitoring. The communication unit 7 can further be specified as wired or wireless connection. Possible implementation including electrically connected wires, traces, or cables, wireless technologies known to persons skilled in the art including and not limited to Bluetooth®, Wi-Fi, ZigBee, optical, acoustic, photoacoustic, and other communication technologies.

Referring to FIG. 2, the communication unit 7 can be used to communicate the control signals for the light emitting diodes (LED) including and not limited to the intensity, duration, intensity pattern, timing of the on/off status of the LEDs. Synchronization of the different sensing modules and the control unit can be achieved through the communication unit 7. The location of the communication unit 7 in the figure is simply for illustration purposes and can be at other locations.

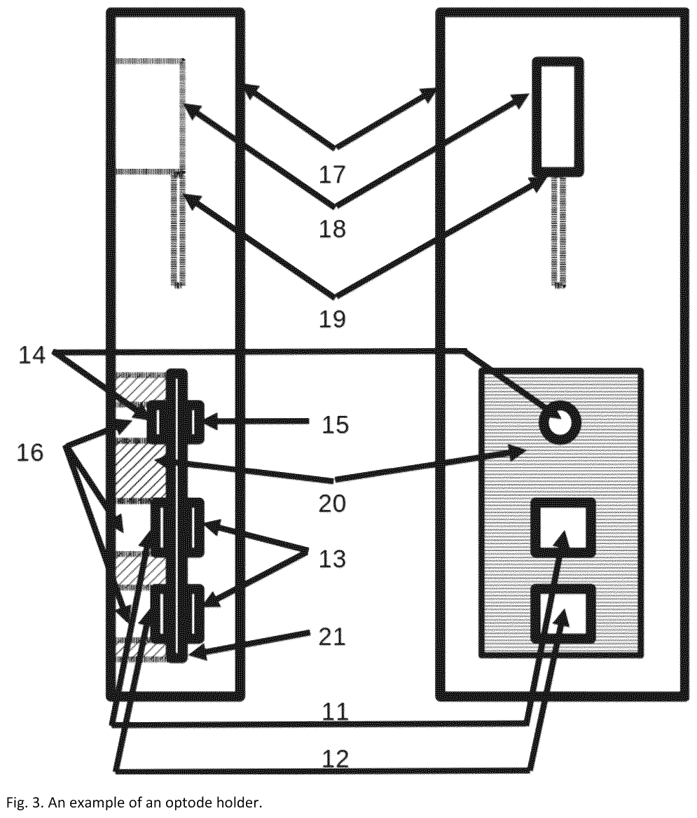

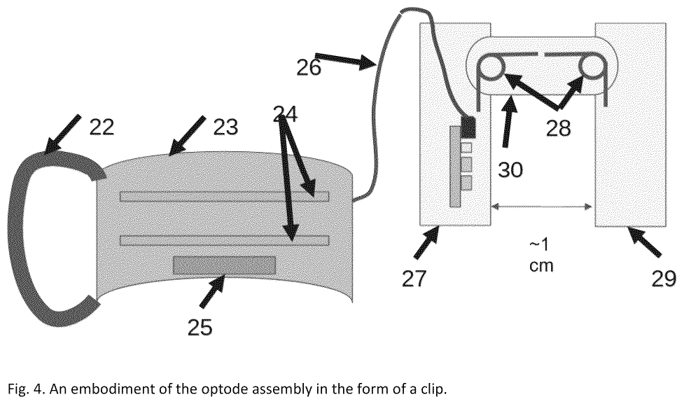

FIG. 3 shows one embodiment of the optode holder. The left panel shows the cross-section side view of the holder 17. The holder is attached to the rest of the optode assembly, as illustrated in FIG. 4, and enabled to firmly and gently adapt to the varying dimension of a penis via a spring mechanism. In other embodiments, other mechanisms to securely attach the optode to the penis can be made with double sided tape, elastic restrictions such as elastic tape or bands. The implementation can be accomplished by persons skilled in the art after this disclosure.

Referring to FIG. 3, in one embodiment, the springs fit into the slots 18, 19 designed to hold the spring in place. An optically non-reflecting mask 20 is used to ensure there is no undesired optical cross-interference among the photodiodes and further ensures that probing light from the LEDs 14 do not have a path reaching the photodiodes 11, 12 without going through the probed tissue first. On the other hand, the openings 16 on the mask 20 allow probing light that has gone through the probed tissue to reach and be measured by the photodiodes 11, 12, converted and amplified by the amplifiers 13, and transmitted out through the communication unit 15 from the optode circuit 21.

FIG. 4 shows a preferred embodiment of the optode assembly in the form of a clip. The clip comprises two legs 27, 29 connected with a bridge 30 with embedded torsion springs 28 of the proper stiffness to ensure comfort and fit. The leg 27 shown to the left of the bridge 30 is illustrated with an embodiment of the optode holder, as shown in FIG. 3. The shown embodiment does not show electronics inside the right leg 29, which is not to be interpreted as a recommendation but only as a preferred embodiment for the purpose of this disclosure. The clip allows rotational degrees of freedom to allow dynamic adaptation to the changing size of the penis. Further, the embodiment will allow a single mechanism to fit a wide range of penis sizes without modifications to the structure of the holder. To further increase the applicable range of penis size or to increase the pressure applied to the tissue to assure desired optical coupling, varying strength or number of springs can be used, and such adaptations are known to persons skilled in the art. In addition, the bridge 30 length can be adjusted for the more permanent adaptation for different ranges of penis sizes. In other embodiments, other mechanisms to securely attach the optode to the penis can be made with double sided tape, elastic restrictions such as elastic tape or bands. The implementation can be accomplished by persons skilled in the art after this disclosure.

Referring to FIG. 4, in the preferred embodiment. The optode assembly (right half of the diagram) is connected to the control unit 23 via a cable 26. In a preferred embodiment, this can be readily replaced by other wired connection technologies. In other embodiments, it can be replaced with wireless technology. In the preferred embodiment, digital signals are used for improved ambient noise suppression. Both analog or digital signals can be transmitted via the cable 26 or compatible technologies. The housing for the control unit 23 can be constructed with different materials depending on the application. In the preferred embodiment, medical grade plastic is used to form a water and disinfectant resistant barrier to the exterior. Furthermore, medical grade plastic is used to ensure safety, reliability, and comparability with the transmission of wireless signals. In the preferred embodiment, a chain 22 is installed on the side of the control unit 23 to allow the unit to be hung on the side. The chain can be missing in other embodiments. Further, the preferred embodiment includes a concave shape on the bottom to allow the control box 23 the flexibility to be placed on a person's leg. This feature can also be missing in other embodiments. The main function of the control box 23 is to house the control, data processing, communication electronics 24 in a safe, reliable, and convenient fashion while offloading the weight and bulkiness of components that must make contact with the patients. In the preferred embodiment, the control unit 23 also houses a battery pack 25 to allow battery operation. Charging will also be performed via the control unit 23 via technologies known by persons skilled in the art including using a USB port or wireless inductive charging.

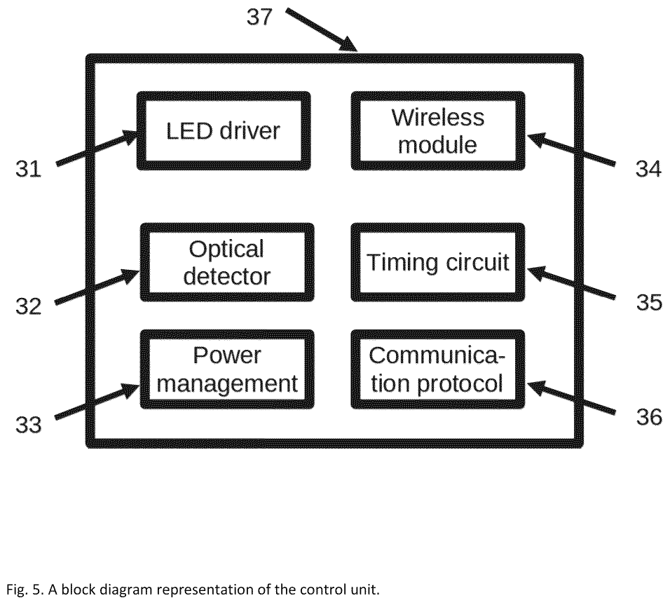

FIG. 5 shows a block diagram representation of the control unit 37. A preferred embodiment of the control unit consists of a LED driver 31, optical detector 32, power management 33, wireless module 34, timing circuit 35, and communication protocol 36. In other embodiments, the control unit can serve a smaller or larger set of features. The LED driver 31 is to control the timing of the red and near infrared LEDs. In the preferred embodiment, the red and near infrared LEDs are set to turn on periodically alternatively. The LED driver can further be used for timing purposes to share the same optical detection system for both the red and near infrared signals. The optical detector 32 is to convert the analog optical signal coming from the optode assemblies into digital signal. There are various methods to accomplish this task that are commonly referred to as analog-to-digital converters. The power management 33 is to source from the available power source, a rechargeable battery in the case of the preferred embodiment, and regulate and condition the power before supplying it to the rest of the electronics. Further, the power management in the preferred embodiment includes a rechargeable battery that can provide enough power to cover the duration of the anticipated operation after taking expected aging into consideration. The wireless module 34 is to provide a means to wirelessly communicate with external devices. In the preferred embodiment, features supported by the wireless module include data transmission, sensing module controls (e.g., EMG and optodes), application configuration, warning signals, battery status, etc. The timing circuit 35 is to allow synchronization of the various modules and allow proper allocation of resources to the various clients that the control unit is serving. The communication protocol 36 ensures proper communication to take place, establishes secure connections when necessary, and allows proper authorizations.

One preferred embodiment monitors hemodynamics with the optode assembly targeting the Corpora Cavernosa regions. The optode assembly consists of two LEDs of red and near infrared wavelengths, and photodiodes at predefined distances. The photodiode closer to the LEDs will detect light that probes the shallower region while the further photodiode will detect light that probes the deeper regions. The red and near infrared wavelengths are used as the probing light as oxyhemoglobins and deoxyhemoglobins have opposite relative optical absorbance between the two wavelengths. The red and near infrared blink alternatively at a fixed rate. When the red LED is on, the photodiode in the optode assembly will measure the red light and vice versa. By using this scheme, the relative concentrations of oxyhemoglobin, deoxyhemoglobin, and total hemoglobins can be measured via modified Beer-Lambert law, which in turn correlates with penile blood flow. Monitoring of penile blood flow before, during, and after stimulation (e.g., tetanic stimulation of the S2-S4 nerve roots to increase the blood flow) will allow the monitoring of penile function. The disclosed technology can monitor the intensity and speed of the change in penile blood flow to allow the development of pathological patterns and identify nerve irritation and vascular irritation in real-time when combined with EMG monitors and machine learning techniques.

By following the above-listed steps, penile blood flow can be measured for the monitoring of neurological and vascular function of structures at surgical risk. This disclosure is particularly advantageous in continuous, autonomous monitoring with real-time feedback to surgeons for potential intervention and decision-making in a timely manner. The measurements result can be used to provide real-time feedback to surgeons operating on the patient, or for off-line analysis and evaluation. The preferred embodiment can operate autonomously in the continuous measurement mode, a synchronized mode, or a triggered mode, reducing the need of highly-skilled apparatus operators, allowing automatic and real-time warning to surgeons when operating in areas while preserving the functions of critical nerves, resulting in improved clinical outcomes and reducing operation costs.

### SOFTWARE

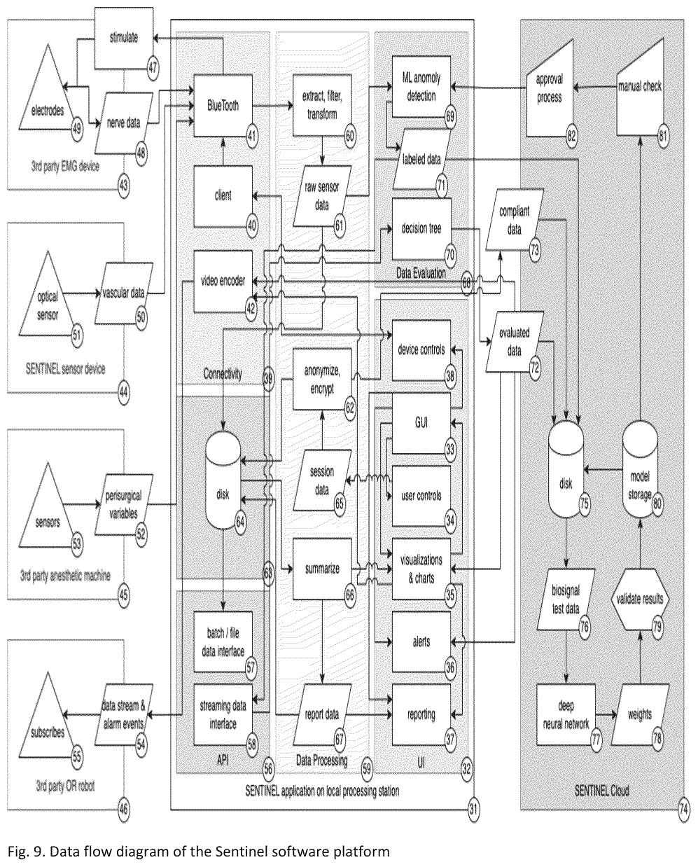

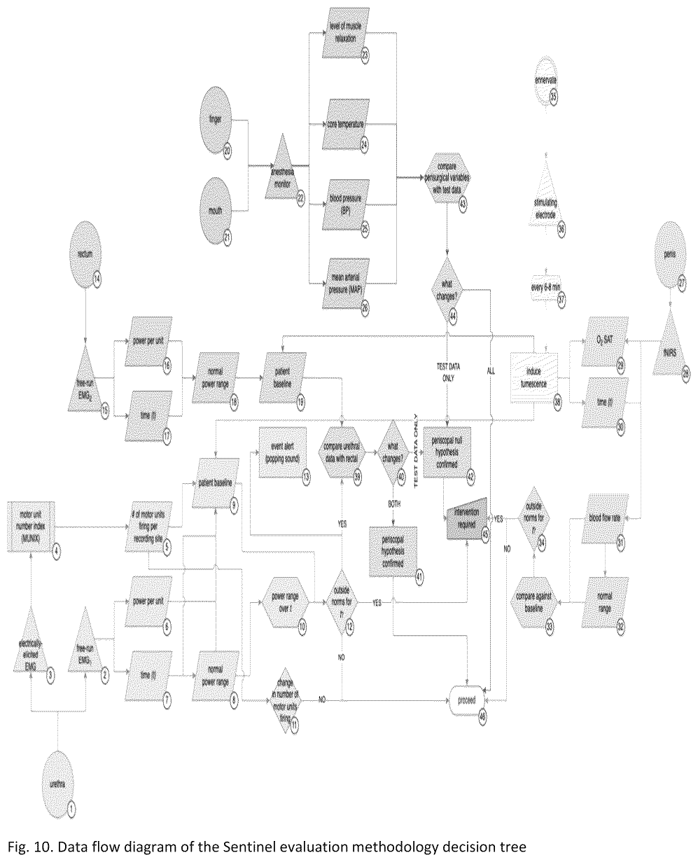

Software components for certain embodiments are described with reference to FIGS. 9-10 in which the following reference numerals may be used:
31. Sentinel application
32. UI module
33. Browser GUI
34. User controls
35. Visualizations and charts
36. Alerts
37. Reporting interface
38. Device controls
39. Connectivity module
40. Connectivity client
41. BlueTooth client
42. Video encoder
43. 3rd party EMG device
44. Sentinel fNIRS sensor device
45. 3rd party anesthetic machine
46. 3rd party OR robot
47. Stimulating function
48. EMG data
49. electrodes
50. Vascular data
51. Optical fNIRS sensor
52. Perisurgical variables
53. Anesthesia equipment sensors
54. Data stream and alarm events
55. Subscription module of OR robot
56. API
57. Batch/file endpoints
58. Streaming data interface
59. Data processing module
50. Extract, Filter, and Transform functions
61. Raw (preprocessed) sensor data
62. Compliance module (anonymization and encryption)
63. Storage module
64. Storage disk
65. Session data
66. Summarization functions
67. Report data
68. Data evaluation (AI) module
69. ML anomaly detection
70. Decision tree
71. Labeled data
72. Evaluated data
73. Compliant (meta) data
74. Sentinel Cloud
75. Cloud disk
76. Biosignal test data
77. Deep neural network
78. Weights
79. Results validation functions
80. Model storage
81. Manual checks
82. Approval and release process
83. External urethral sphincter recording site
84. Free-run EMG via electrode
85. Electrically-elicited EMG
86. Motor Unit Number Estimation function
87. Data output of count of motor units firing per recording site
88. Data output of power per unit measurement
89. Data output of time measurement
90. Data output of normal power range calculation
91. Data output of patient baseline characteristics
92. Data output of power range over time calculation
93. Function for logic gate if there is a change in number of motor units firing
94. Function for logic gate if input is outside normative range for a given time
95. Audible event; popping sound of nerves firing
96. Rectal sphincter recording site
97. Free-run EMG via electrode
98. Data output of power per unit measurement
99. Data output of time measurement
100. Data output of normal power range calculation
101. Data output of patient baseline characteristics
102. Finger recording site
103. Mouth recording site
104. Anesthesia patient monitor
105. Data output of level of muscle relaxation measurement
106. Data output of patient core temperature measurement
107. Data output of blood pressure measurement
108. Data output of mean arterial pressure measurement
109. Penis recording site
110. fNIRS sensor
111. Data output of blood oxygen saturation measurement
112. Data output of time measurement
113. Data output of blood flow rate measurement
114. Data output of normal range of variability measurement
115. Function to check input against patient's baseline
116. Function for logic gate if input is outside normative range for a given time
117. Sacral nerve stimulation event
118. Stimulating electrode
119. Frequency of stimulation
120. Induced tumescence
121. Function to compare urethral data against rectal data
122. Function for logic gate to determine outputs based on inputs
123. Periscopal hypothesis confirmation
124. Periscopal null hypothesis confirmation
125. Function to compare perisurgical variables against test data
126. Function for logic gate to determine outputs based on inputs
127. Determination that intervention is necessary
128. Determination that it is safe to proceed; that no intervention is necessary.

The following are additional details of the reference numerals in accordance with certain embodiments:
31. Sentinel application runs on a local processing station (e.g., a laptop) and comprises functional modules for real time data processing, evaluation, visualization, reporting, and device operations.
32. UI module contains functionality to operate and administer the Sentinel software.
33. Browser GUI shows reporting and data visualizations and enables alerting, device, user management, and session operations.
34. User controls via the Browser GUI enables user profile and session management.
35. Visualizations and charts via the Browser GUI shows summary statistics, charts, and other visualizations.
36. Alerts are audio and visual, and optionally tactile, and can be managed via the Browser GUI.
37. Reporting interface shows report data and enables saving and retrieving.
38. Device controls enable management of connected devices.
39. Connectivity module contains functions to manage connected devices through BlueTooth and USB where necessary.
40. Connectivity client enables communication between the Sentinel application and integrated devices.
41. Bluetooth client manages transmission of Bluetooth packets between the application and integrated device.
42. Video encoder prepares relevant data, metrics, and visualizations for video output.
43. 3rd party EMG device enables stimulating electrodes and the collection of EMG data.
44. Sentinel fNIRS sensor device enables collection of blood oxygenation data.
45. 3rd party anesthetic machine enables collection of patient data to control for perisurgical variables.
46. 3rd party OR robot can collect, utilize, and/or display Sentinel-processed data.
47. Stimulating function enables stimulating nerves to elicit responses.
48. EMG data enables processing of various tests, including MUNE, FEMG, TEMG, SEP, and MEP.
49. Electrodes collect EMG data.
50. Vascular data is blood oxygenation data collected by the Sentinel vascular sensor.
51. Optical fNIRS sensor collects vascular data.
52. Perisurgical variables include the patient's breathing, heart rate, and related systemic data.
53. Anesthesia equipment sensors enable the collection of perisurgical data.
54. Data stream and alarm events from Sentinel can be consumed by the OR robot.
55. Subscription functions of OR robot enable ingestion of Sentinel data.
56. API enables machine to machine communication with the Sentinel platform.
57. Batch/files endpoints enable specific methods used to retrieve or update data from and to the Sentinel platform.
58. Streaming data interface enables external consumption of labeled data and adding decision trees per use case.
59. Data processing module contains functions to manipulate, sanitize, prepare inbound data for evaluation, and summarize resulting data for storage, display, and reporting.
60. Extract, Filter, and Transform functions prepare inbound data for processing.
61. Raw (preprocessed) sensor data can be sanitized and processed by the Data Evaluation module.
62. Compliance module anonymizes and encrypts data.
63. Storage module processes the receipt and storage of data from other modules.
64. Storage disk provides local storage as part of the Storage module.
65. Session data is collected from the Browser GUI and stored to local disk.
66. Summarization functions take stored processed and evaluated data to generate metrics and charts, as well as populate reporting data.
67. Report data contains session information and summary statistics.
68. Data evaluation (AI) module, consisting of the ML model and decision tree, processes sensor data to determine onset of true positive nerve irritation or injury.
69. ML anomaly detection is trained to detect relevant events.
70. Decision tree processes events to determine the onset of true positive nerve irritation or injury.
71. Labeled data outputs from the ML model can be ingested by different decision trees and is exposed by the streaming data interface.
72. Evaluated data from the decision tree is stored to both local and cloud disk, and can be used for charts and visualizations, as well as to inform alerts.
73. Compliant (meta) data contains relevant patient metadata to enable extra-operative analysis.
74. Sentinel Cloud contains remote server functionality, including storage and model training.
75. Cloud disk stores locally evaluated data.
76. Biosignal test data collected from evaluated data enables further ML training.
77. Deep neural network trains the model for improved event detection.
78. Weights are outputs from the deep neural network used to adjust the ML model.
79. Results validation functions test weights for improved event detection.
80. Model storage stores model iterations and weights.
81. Manual checks by software engineers provide additional validation before distribution of software updates.
82. Approval and release process ensures safe distribution of software updates.
83. External urethral sphincter recording site provides EMG data through electrodes.
84. Free-run EMG via electrodes at the recording site (urethra) provides measurement data for various tests.
85. Electrically-elicited EMG enables tests like Motor Unit Number Estimation.
86. Motor Unit Number Estimation function outputs counts of motor units firing per recording site.
87. Data output of count of motor units firing per recording site provides basis for measuring subsequent changes.
88. Data output of power per unit measurement and/or other EMG activities such as spikes, bursts, and neurotonic discharges provide basis for measuring subsequent changes.
89. Data output of time measurement provides time and/or frequency domain analysis.
90. Data output of normal power range calculation and/or other EMG activities detection provide basis for measuring subsequent changes.
91. Data output of patient baseline characteristics provides basis for measuring subsequent changes.
92. Function for calculation of power range over time measurement and/or EMG feature identification provide real time comparison against baseline values.
93. Function for logic gate if there is a change in number of motor units firing determines the need for alarm.
94. Function for logic gate if input is outside normative range for a given time determines the need for alarm.
95. Audible event; popping sound of nerves firing as a result of input being outside normative range.
96. Rectal sphincter recording site provides EMG data through electrodes.
97. Free-run EMG via electrode at the recording site (rectum) provides measurement data for various tests.
98. Data output of power per unit measurement and pattern provide basis for measuring subsequent changes.
99. Data output of time measurement provides time and/or frequency domain analysis.
100. Data output of normal power range calculation and pattern provide basis for measuring subsequent changes.
101. Data output of patient baseline characteristics provides basis for measuring subsequent changes.
102. Finger recording site provides patient data through patient monitoring device sensors.
103. Mouth recording site provides patient data through patient monitoring device sensors.
104. Anesthesia patient monitor provides patient data for analysis.
105. Data output of level of muscle relaxation measurement provides patient data as a control for comparison against test data.
106. Data output of patient core temperature measurement provides patient data as a control for comparison against test data.
107. Data output of blood pressure measurement provides patient data as a control for comparison against test data.
108. Data output of mean arterial pressure measurement provides patient data as a control for comparison against test data.
109. Penis recording site provides blood oxygenation data through fNIRS sensor.
110. fNIRS sensor provides blood oxygenation data for subsequent analysis.
111. Data output of blood oxygen saturation measurement provides basis for subsequent tests.
112. Data output of time measurement provides basis for time and/or frequency domain analysis.
113. Data output of blood flow rate measurement, when compared against the patient's own baseline, provides basis for measuring onset of changes.
114. Data output of normal range of variability measurement provides patient's own baseline as basis for comparison.
115. Function to check input against patient's baseline to determine the need for alarm.
116. Function for logic gate if input is outside normative range for a given time to determine the need for alarm.
117. Sacral nerve stimulation event is triggered by the stimulating electrode.
118. Stimulating electrode enervates nerves to elicit a measurable response.
119. Frequency of stimulation can be determined by the operator.
120. Induced tumescence events can be measured by the fNIRS sensor and provide validation of otherwise measured events.
121. Function to compare urethral data against rectal data can be used to test the periscopal hypothesis in some embodiments.
122. Function for logic gate to determine outputs based on inputs based on whether urethral and rectal data change in some embodiments.
123. Periscopal hypothesis confirmation event in case both urethral and rectal data change.
124. Periscopal null hypothesis confirmation event in case urethral data change only.
125. Function to compare perisurgical variables against test data enables testing for the periscopal hypothesis.
126. Function for logic gate to determine outputs based on inputs based on whether urethral and patient monitoring data change.
127. Determination that intervention is necessary is based on output of logic gates.
128. Determination that it is safe to proceed; that no intervention is necessary is based on output of logic gates.

In certain embodiments, when the patient comes into the OR, they are connected to an anesthesia patient monitor, an electromyography (EMG) device which measures muscle response or electrical activity in response to a nerve's stimulation of the muscle, and the Functional Near-Infrared Spectroscopy (fNIRS) device that uses near-infrared light to monitor blood flow, across multiple recording sites. If there is an operating room (OR) robot or similar surgical platform, the invention can also be connected to it to transmit data. An authorized individual ensures the connection of these various devices to the local processing station on which the software is running (e.g., a laptop), then starts the session. Sentinel will then initiate a test called Motor Unit Number Estimation (MUNE), which estimates the number of motor units firing when a muscle contracts. By using ML, Sentinel is able to establish the patient's baseline motor status before surgery begins, which correlates with the patient's level of continence. The software also initiates the Free-run EMG (fEMG) protocol, which calculates the amount of power per unit time and/or identifies EMG activity features. When, for instance, during surgery, the invention has been continuously collecting data and the surgeon is prepping and opening the incision, the invention will calculate normal power range measurements and/or identify baseline pattern from the Free-run EMG (fEMG). When the surgeon is operating and starts to irritate motor fibers, the invention will begin to observe EMG activity. If the activity stays within the normal power range and no abnormal activity patterns are detected, there is no need for intervention; if it falls outside of the range or abnormal patterns are detected, the invention will identify and log the event. The surgeon may hear the popping noise associated with this firing. The software will measure the power over time and if it does not fall back within normal limits within a configurable number of seconds or if the abnormal patterns persist, the software will re-enable the MUNE program. Free-run EMG will still be collected, but the MUNE will determine if there is a change in the number of motor units firing. If so, intervention is initiated and MUNEs continue to run continuously. If the number of units is unchanged, then the surgeon may elect to wait until firing stops (while still running MUNE) and no further intervention is needed. To rule out perisurgical variables, as soon as the software observes increased power, certain embodiments could compare urethral with rectal data. If both change, then the change is likely perisurgical in origin (for example, irrigating the field with cold water) and, for example, would not trigger an alarm; in certain embodiments, if only urethral data change, the software will trigger an alarm.

In certain embodiments, abnormal ranges may be determined for F-EMGs based on the amount of voltage/power over time and the firing patterns; for T-EMG based on an increase in latency of 10% and/or a decrease in amplitude of 50%; for fNIRS based on a change of 20%; and for MUNE a difference of 50%, although different embodiments may determine normal vs. abnormal ranges in other ways include through the use of machine learning.

The decisioning for the alarm, and specifically, and the consideration of perisurgical variables additionally consider two other data sources. During surgery, and particularly during those procedures known to present risk to nerve structures, an authorized individual will initiate a nerve stimulation process by means of the software interface to elicit tumescence; this process will happen concurrently with the Free-run EMG and MUNE process described above. At a configurable frequency, or, for example, once every 6-8 minutes, the invention will administer tetanic stimulation of the S2-S4 nerve roots to increase penile blood flow and elicit tumescence. The invention will apply a machine learning-trained algorithm to identify and classify the events that comprise tumescence; by monitoring penile blood flow before, during, and after stimulation, the invention will allow the monitoring of penile vascular function, providing an additional means of validating that tumescence can be achieved. At the same time, the invention will collect and process perioperative data from the anesthesia patient monitor in real time; these data will include the level of muscle relaxation, core temperature, blood pressure, and mean arterial pressure. By continually comparing test data against these perisurgical variables, the invention will determine whether a change in test data occurred independently of perisurgical data.

The following disclosure is for a software platform for the automated monitoring of neurological and vascular function of structures at surgical risk. The monitoring principle is to identify and evaluate relevant events from streams of physiological data to determine true positive onset of nerve irritation. The preferred embodiment uses a machine learning model to identify relevant events and a decision tree algorithm to compare the data against the patient's baseline to determine the results in real time.

This disclosure is particularly advantageous in continuous, autonomous monitoring with real-time feedback to surgeons for potential intervention and decision-making in a timely manner. The measurements results can be used to provide real-time feedback to surgeons operating on the patient, or for off-line analysis and evaluation. The preferred embodiment can operate autonomously in the continuous measurement mode, a synchronized mode, or a triggered mode, reducing the need of highly-skilled apparatus operators, allowing automatic and real-time warning to surgeons when operating in areas while preserving the functions of critical nerves, resulting in improved clinical outcomes and reducing operation costs.

Data collected and processed using the disclosed invention demonstrates its feasibility and reproducibility. FIG. 6 shows the absorption data of changes in deoxyhemoglobin relative concentration in penile blood. The rectangular boxes indicate periods of tumescence. FIG. 7 shows the absorption data of changes in oxyhemoglobin relative concentration in penile blood. The rectangular boxes indicate periods of tumescence. The changes shown in FIGS. 6-7 agree with the expectation mentioned previously. FIG. 8 shows the overlay of three normalized test runs. It can be seen in the figure that the hemoglobin concentration changes are reproducible and are advantageous to be used as indicators of penile functions.

FIG. 9 shows a data flow diagram representation of the Sentinel application 31, Sentinel Cloud 74, the 3rd party EMG device 43, the Sentinel sensor device 44, the 3rd party anesthesia equipment 45, and the 3rd party OR robot 46. A preferred embodiment of the Sentinel application consists of a UI 32, Data Evaluation module 68, Data processing module 59, Connectivity module 39, Storage module 63, and API 56. In other embodiments, the application can serve a smaller or larger set of features. The Sentinel application runs on a local processing station, like a laptop or the computing unit of an OR surgical robot 46.

In one preferred embodiment, the application UI 32 provides the means of interfacing with the application via the graphical user interface (GUI) 33, which shows the reporting 37 and data visualizations 35 features and enables alerting 36, device control 38, user management 34, and session operations. The user controls 34 enable users to create profiles that describe attributes of the surgery, surgeon, and patient pathology, which then become the source of the session data 65 that is anonymized and encrypted to be written to the Storage module 63. The data visualizations and charts 35 function generates charts and displays summary statistics, which are customizable in view through selection operations to enable displaying data most relevant to the users. The alerts 36 produced and managed by the Browser GUI are both audio and visual in nature. The data visualizations and charts, as well as the summary statistics prepared as report data 67 are aggregated and presented in the Reporting interface 37 for saving and retrieval. The device controls 38 enable the management of the connected devices. In other embodiments, the UI doesn't require the reporting function.

In certain embodiments, the connectivity module 39 contains functions to manage connected devices through wired and wireless communication protocols. In the preferred embodiment, BlueTooth 41 and USB provide both capabilities. In another embodiment, a video encoder 42 prepares visualizations of data, alerts, and metrics for consumption by an external device, like an OR robot. The device controls 38 interact with the connectivity client 40 to transmit and receive data from the integrated devices.

The 3rd party EMG device 43 enables stimulating electrodes and the collection of EMG data, the Sentinel fNIRS sensor device 44 enables collection of blood oxygenation data, and the 3rd party anesthetic machine's 45 sensors enable collection of patient data to control for perisurgical variables. Perisurgical variables 52 include the patient's level of muscle relaxation, core temperature, blood pressure, and mean arterial pressure. The stimulating electrodes 47 of the EMG device 43 enables stimulating nerves to elicit responses, and electrodes collect EMG data 48, which enables the processing of various tests, including MUNE/MUNIX, FEMG, TEMG, SEP, and MEP. The Sentinel sensor device's optical fNIRS sensor 51 collects vascular data 50, or blood flow and oxygen saturation data. In one embodiment, the 3rd party OR robot 46, which can collect, utilize, and/or display Sentinel-processed data, integrates with the Sentinel platform to consume video data 54 via a video input subscription service 55.

Data received from the integrated devices is processed by the data processing module 59, which in any embodiment, include software functions that extract, filter, and transform 60 in real time by means known to those of the art. This process produces sensor data 61. The data processing functions also include anonymization and encryption functions 62 in keeping with regulatory requirements, and summarization functions 35 that take stored processed and evaluated data to generate metrics and charts, as well as populate reporting data 66, or session information and summary statistics. The anonymization function also collects session data 65 from the Browser GUI to process the data and store it to local disk 64.

In one preferred embodiment, sensor data 61 is sent to the storage 63 and data evaluation 68 modules, where a storage mechanism known to those in the art stores the data to disc 64; the disc refers to the local storage drive available to the application. The data is then retrievable by other functions, including the summarization function and the data evaluation module. A streaming data interface enables real time subscription to the labeled data outputs 71 from the ML model 69. In other embodiments, user data and report data, among others, are accessible via the API 56 (by means of a protocol like REST, or any other mechanism known to those in the arts that enable specific methods used to retrieve or update data from and to the Sentinel platform), or a similar means of exposing structured data.

In certain embodiments, sensor data 61 that is sent to the data evaluation module 68 is first processed by the machine learning-trained model 69 to detect and classify events; labeled events 71 are then compared against the patient's baseline and against other indications via the decision tree 70 to determine the true positive onset of nerve irritation. The decision tree represents the logic of the proprietary IOM methodology, as shown in FIG. 10. Evaluated data 72 from the decision tree is stored to both local and cloud disks, and can be used for charts and visualizations, as well as to inform alerts. The cloud disk 75 is storage provisioned in a remote server, which, along with other remote servers and the functions running on them, comprise the Sentinel Cloud 74. Along with the evaluated data, compliant (meta) data 73 that contains relevant patient, surgeon, and OR metadata to enable extra-operative analysis is sent from the compliance function to the cloud disk.

The cloud disk stores and makes available biosignal test data 76 collected from evaluated data, which enables further ML training; a deep neural network 77 trains the model for improved event detection. Weights 78, used to adjust the distributed version of the ML model, are outputted from the deep neural network, and results validation functions 79 test these weights for improved performance. Model storage 80 is another storage disk that then stores model iterations and weights. Manual checks 81 by software engineers provide additional validation before the distribution of the model and other software updates. Approval and release processes 82 ensure safe distribution of software updates.

In certain embodiments, the EMG 43 (FIG. 9) will run free-run EMGs 84,97 continuously, recording from the external urethral sphincter 83 and the rectal sphincter 96, as shown in FIG. 10. Free-run EMG produces power per unit data 88,98 (and can also include other EMG activity features as outlined above), which can be measured over time 89,99. This resulting normal power range data 100 collected from the rectal sphincter will act as the control for perisurgical variables, against the test normal power range data 90 from the urethra, which then also informs the patient baseline 91,101. A subsequent function compares the continual power per unit data against the patient baseline 92 in real time, the output of which is then analyzed by a logic gate function 94; if the output is outside the normative range across time, certain embodiments trigger the function to compare urethral data against rectal data 121 to test for the periscopal hypothesis. In the event that the continual power per unit is measured to be outside the normal range, there may also be a corresponding audible event 95 in which the popping sound of nerves firing can be heard.

In certain embodiments, in addition to the Free-run EMG, electrically-elicited EMG 85 enables tests like Motor Unit Number Estimation; the function that enables Motor Unit Number Estimation 86 outputs counts of motor units firing per recording site. These continual counts of units firing per recording site 87 provides the basis for the function that calculates subsequent changes in the number of motor units firing 93; if there is no change, the invention will not trigger an alarm 128.

To validate a true positive event as detected by means of EMG, certain embodiments can include a function that compares urethral data against rectal data 121 to test for the periscopal hypothesis. A subsequent logic gate function 122 could test for the periscopal hypothesis 123 based on inputs based on whether urethral and rectal data change. The periscopal null hypothesis is confirmed 124 in the case that urethral data change only. Similarly, a function that compares perisurgical variables against test data 125 enables additional testing for the periscopal hypothesis. Here too, a logic gate function 126 tests for the persiscopal hypothesis based on inputs whether urethral and patient monitoring data change. When test data change only, the invention determines that intervention is necessary 127; likewise, if no or all data change, the invention determines that it is safe to proceed 128 and that no intervention is necessary.

In one preferred embodiment, an anesthesia or patient monitor device 104 provides systemic patient health data, including measurements of: level of muscle relaxation 105; patient core temperature 106; blood pressure 107; and mean arterial pressure 108 as controls for comparison against test data. These data are collected from the relevant recording sites (i.e., finger, mouth) 102,103.

In one preferred embodiment, blood oxygenation data 111 across time measurements 112 is retrieved from the penis 109 recording site by means of a fNIRS sensor 110. Changes in blood flow rate measurements 113, elicited by the inducement of tumescence 120, for example, provides the basis for validating the onset of change in data. Tumescence is induced by sacral nerve stimulation 117 by means of the stimulating electrode 118. The frequency of stimulation 119 can be determined by the operator. The data output of the calculated normal range of variability 114 enables the use of the patient's own baseline as the basis for comparison; this function checks the data against the patient's baseline 115. Subsequently, a logic gate function 116 checks if data are outside the normative range for a given time to determine the need for alarm.

### Miscellaneous

It should be noted that headings are used above for convenience and are not to be construed as limiting the present invention in any way. The section under the heading HARDWARE focuses on hardware used in various penile blood flow monitoring embodiments and the section under the heading SOFTWARE focuses on software used in various penile blood flow monitoring embodiments. Each section is intended to be self-contained and internally consistent with respect to terminology, reference numerals, etc., although the two sections are intended to be complementary to one another (e.g., hardware described in the HARDWARE section may be used to implement some or all methodology described in the SOFTWARE section and some or all of the methodology described in the SOFTWARE section may be implemented using hardware described in the HARDWARE section). The two sections may use different terminology or reference numerals to refer to a common or similar concept or component.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Although the above discussion discloses various exemplary embodiments of the invention, it should be apparent that those skilled in the art can make various modifications that will achieve some of the advantages of the invention without departing from the true scope of the invention. Any references to the “invention” are intended to refer to exemplary embodiments of the invention and should not be construed to refer to all embodiments of the invention unless the context otherwise requires. The described embodiments are to be considered in all respects only as illustrative and not restrictive.

1. An optical penile structure function monitoring system comprising:
a measuring probe configured to be secured via an attachment mechanism to a penis of a patient during and over tumescence and flaccid penile events; and
a control system comprising:
a first detector that detects penile blood flow based on signals received from the measuring probe; and
a second detector that detects tumescence and flaccid penile events based on signals received from the first detector.

2. The system of claim 1, wherein at least one of:
the measuring probe comprises at least one optode assembly of optical components to measure penile blood flow hemodynamics;
the measuring probe and control system are configured to measure optical absorbance signals with at least one wavelength and optionally with at least two wavelengths;
the measuring probe comprises a compressible optically non-reflective mask to separate at least two optical components;
the measuring probe is configured to convert optical signals to electrical signals, and wherein the control system is configured to process the electrical signals from the measuring probe and to record continuous raw data and penile function data;
the measuring probe has a synchronization mechanism to allow the sharing of optical components; or
at least one of the measuring probe or the control system is configured to provide visual guidance to configure the system according to the surgery type.

3. (canceled)

4. (canceled)

5. (canceled)

6. (canceled)

7. The system of claim 1, wherein the control system is configured to provide in-time warnings to surgeons operating on a patient who might have their penile function at risk based on the signals received from the measuring probe, optionally wherein at least one of:
the in-time warnings include at least one of a visually warning or an audible warning; or
the control system includes a memory to record any warnings and their associated measurements.

8. The system of claim 1, wherein the attachment mechanism is configured to adapt to different penile sizes using the same hardware, optionally wherein the attachment mechanism comprises a penile clip and/or double-sided tape.

9. (canceled)

10. (canceled)

11. The system of claim 1, wherein the control system includes a control unit separate from the measuring probe, optionally wherein communication between the control unit and the measuring probe comprises flexible shielded cables or a wireless connection.

12. (canceled)

13. (canceled)

14. The system of claim 7, wherein normal ranges of variability, thresholds, and types of significant data changes as a function of surgical maneuver or other physiological parameters are established and stored, optionally wherein at least one of (a) the normal ranges, thresholds, and types of significant data changes are established using machine learning, or (b) the in-time warnings are generated using machine learning.

15. (canceled)

16. (canceled)

17. (canceled)

18. (canceled)

19. (canceled)

20. The system of claim 2, wherein the at least two wavelengths include red and near infrared wavelengths, optionally wherein the red and near infrared wavelengths are used respectively as the probing light for oxyhemoglobins and deoxyhemoglobins that have opposite relative optical absorbance between the two wavelengths.

21. (canceled)

22. The system of claim 20, wherein measurements alternate between the red and near infrared wavelengths, optionally wherein the measurements alternate at a fixed rate.

23. (canceled)

24. The system of claim 1, further comprising an EMG monitor that measures muscle response or electrical activity in response to stimulation of a nerve of the patient, wherein the control system is configured to monitor the intensity and speed of the change in penile blood flow to develop pathological patterns and identify nerve irritation and vascular irritation in real-time when combined with signals from the EMG monitor, optionally wherein the control system is further configured to develop pathological patterns and identify nerve irritation and vascular irritation in real-time using machine learning.

25. (canceled)

26. The system of claim 1, wherein the measuring probe comprises at least one LED capable of producing red wavelength light and near infrared wavelength light and further comprises at least one photodiode capable of measuring red wavelength light and near infrared wavelength light, optionally wherein at least one of:
the at least one LED comprises at least one red wavelength LED and at least one near infrared wavelength LED;
the measuring probe comprises at least one optically non-reflecting mask configured to prevent undesired optical cross-interference between the at least one LED and the at least one photodiode and further configured to ensure that probing light from the at least one LED does not have a path for reaching the at least one photodiode without going through the probed tissue first; or
the measuring probe is configured or controllable to alternate between producing red wavelength light and producing near infrared wavelength light.

27. (canceled)

28. The system of claim 26, wherein at least one of:
the at least one photodiode comprises at least one red wavelength photodiode and at least one near infrared photodiode;
the at least one photodiode comprises a single photodiode used to detect both the red wavelength light and the near infrared wavelength light; or
the at least one photodiode comprises at least a first photodiode placed closer to the red and near infrared LEDs for detecting light that probes a shallower region and a second photodiode placed further from the red and near infrared LEDs for detecting light that probes a deeper region.

29. (canceled)

30. (canceled)

31. (canceled)

32.

33. A measuring probe comprising:
at least one LED capable of producing red wavelength light and near infrared wavelength light; and
at least one photodiode capable of measuring red wavelength light and near infrared wavelength light.

34. The measuring probe of claim 33, wherein the at least one LED comprises at least one red wavelength LED and at least one near infrared wavelength LED, optionally wherein the measuring probe is configured or controllable to alternate between producing red wavelength light and producing near infrared wavelength light.

35. The measuring probe of claim 33, wherein at least one of:
the at least one photodiode comprises at least one red wavelength photodiode and at least one near infrared photodiode; or
the at least one photodiode comprises a single photodiode used to detect both the red wavelength light and the near infrared wavelength light.

36. (canceled)

37. The measuring probe of claim 33, wherein the at least one photodiode comprises at least a first photodiode placed closer to the red and near infrared LEDs for detecting light that probes a shallower region and a second photodiode placed further from the red and near infrared LEDs for detecting light that probes a deeper region.

38. The measuring probe of claim 33, wherein the measuring probe comprises at least one optically non-reflecting mask configured to prevent undesired optical cross-interference between the at least one LED and the at least one photodiode and further configured to ensure that probing light from the at least one LED does not have a path for reaching the at least one photodiode without going through the probed tissue first.

39. (canceled)

40. The measuring probe of claim 33, further comprising an attachment mechanism configured to secure the measuring probe to a penis of a patient during and over tumescence and flaccid penile events, optionally wherein at least one of:
the attachment mechanism is configured to adapt to different penile sizes using the same hardware; or
the attachment mechanism comprises a penile clip and/or double-sided tape.

41. (canceled)

42. (canceled)

43. (canceled)

44. The measuring probe of claim 33, further comprising at least one of:
a communication interface for wired and/or wireless communication with a control system; or
a synchronization mechanism to allow the sharing of optical components.

45. (canceled)

46. (canceled)

47. A method for monitoring a patient, the method comprising:
monitoring penile blood flow of the patient;
determining a normal range of penile blood flow for the patient;
initiating nerve stimulation to elicit tumescence;
applying a machine learning-trained algorithm to identify and classify penile blood flow in response to the nerve stimulation; and
initiating intervention when the penile blood flow in response to the nerve stimulation is outside of the normal range of penile blood flow for the patient.

48. The method of claim 47, wherein at least one of:
monitoring penile blood flow of the patient comprises monitoring blood oxygenation data; or
initiating nerve stimulation uses a stimulating electrode.

49. (canceled)
